(19) United States
(12) Patent Application Publication
(10) Pub. No.: US 2024/0382128 A1
(43) Pub. Date: Nov. 21, 2024

(54) BIOLOGICAL SIGNAL ANALYZING APPARATUS, WAVEFORM GENERATION IDENTIFICATION METHOD, AND COMPUTER-READABLE MEDIUM

(71) Applicants: Kazuya NIYAGAWA, Kanagawa (JP); Miyako ASAI, Osaka (JP); Masayuki HIRATA, Osaka (JP)

(72) Inventors: Kazuya NIYAGAWA, Kanagawa (JP); Miyako ASAI, Osaka (JP); Masayuki HIRATA, Osaka (JP)

(21) Appl. No.: 18/661,808

(22) Filed: May 13, 2024

(30) Foreign Application Priority Data
May 19, 2023 (JP) 2023-083473

(51) Int. Cl. A61B 5/243

(52) U.S. Cl. CPC A61B 5/243; A61B 2562/0223

(57) ABSTRACT

A biological signal analyzing apparatus includes an acquisition unit, a detection unit, and a search unit. The acquisition unit is configured to acquire waveform data of biological signals measured by a plurality of sensors. The detection unit is configured to detect a time at which characteristic waveform information about an interictal epileptiform discharge (IED) appears in the waveform data acquired by the acquisition unit. The search unit is configured to search for an onset time at which a signal is generated from a lesion in a brain within a predetermined search range based on the time detected by the detection unit in the waveform data.

### CROSS-REFERENCE TO RELATED APPLICATION

The present application claims priority under 35 U.S.C. § 119 to Japanese Patent Application No. 2023-083473, filed on May 19, 2023. The contents of which are incorporated herein by reference in their entirety.

### BACKGROUND OF THE INVENTION

### 1. Field of the Invention

The present invention relates to a biological signal analyzing apparatus, a waveform generation identification method, and a computer-readable medium.

### 2. Description of the Related Art

In the bedside diagnosis of epilepsy using magnetoencephalography and electroencephalography, the localization of epileptic lesions in the brain is evaluated using a technique called an equivalent current dipole method. The equivalent current dipole method estimates the location of the epileptic lesion by estimating the current source (dipole) that produces the magnetic field measured on the scalp. In order to perform the dipole estimation, it is necessary to identify the time at which characteristic waveform information (also called a spike) called an interictal epileptiform discharge (IED) appears from the time series of waveform data detected by multiple sensors, and to identify the sensor on which the waveform information appears.

However, signals generated from epileptic lesions can move with time, and the signal at the time it is generated from the epileptic lesion is not necessarily characterized by a maximum signal intensity. It is thus not necessarily true that the estimated location is the epileptic lesion, though the location is estimated based on the waveform data corresponding to the time at the peak of the characteristic waveform of the IED after the characteristic waveform is detected in the waveform data. It is thus necessary to manually search for the time (onset time) at which the signal is generated from the actual epileptic lesion on the basis of information such as an isomagnetic field diagram retroactively from the peak of the waveform data, which is very laborious. In such manual search, there is also a problem in that the search results may vary depending on the skills of experts, such as doctors.

As a technique for extracting the time at which and the sensor in which the characteristic waveform information appears in the waveform data, a method is disclosed in Japanese Unexamined Patent Application Publication No. 2021-069929, for example. The method uses a comparison step of comparing individual waveform data acquired by multiple sensors with at least one piece of characteristic waveform information, a determination step of determining an appearance probability of the characteristic waveform information in at least a certain section of the waveform data on the basis of a degree of correlation between a peak section of the waveform data and the characteristic waveform information, and an identification step of identifying a time at which a section matching with the characteristic waveform information appears and a concerned sensor on the basis of the appearance probability.

The method described in Japanese Unexamined Patent Application Publication No. 2021-069929 identifies the time on the basis of the appearance probability with respect to the IED. The method, however, often infers the time near the peak of the characteristic waveform of the IED. The problem thus still remains in that the time at which the signal is generated from the epileptic lesion (the onset time) must be manually searched.

In view of the above, the invention aims to provide a biological signal analyzing apparatus, a waveform generation identification method, and a computer program that can automatically identify the time at which a signal is generated from an epileptic lesion, thereby making it possible to achieve efficient diagnosis for epilepsy and to increase the stability of diagnostic accuracy.

### SUMMARY OF THE INVENTION

According to an aspect of the present invention, a biological signal analyzing apparatus includes an acquisition unit, a detection unit, and a search unit. The acquisition unit is configured to acquire waveform data of biological signals measured by a plurality of sensors. The detection unit is configured to detect a time at which characteristic waveform information about an interictal epileptiform discharge (IED) appears in the waveform data acquired by the acquisition unit. The search unit is configured to search for an onset time at which a signal is generated from a lesion in a brain within a predetermined search range based on the time detected by the detection unit in the waveform data.

### BRIEF DESCRIPTION OF THE DRAWINGS

FIG. 1 is a diagram illustrating an exemplary overall structure of a biological signal measurement system according to a first embodiment;

FIG. 2 is a diagram illustrating an exemplary structure of functional blocks of a server according to the first embodiment;

FIG. 3 is a diagram illustrating an exemplary hardware structure of an information processing apparatus according to the first embodiment;

FIG. 4 is a diagram illustrating an exemplary structure of the functional blocks of the information processing apparatus according to the first embodiment;

FIG. 5 is a diagram illustrating exemplary waveform data in which characteristic waveform information about an interictal epileptiform discharge (IED) appears;

FIG. 6 is a flowchart illustrating an exemplary onset search processing flow of the information processing apparatus according to the first embodiment;

FIG. 7 is a flowchart illustrating an exemplary origin-peak determination processing flow of the information processing apparatus according to the first embodiment;

FIG. 8 is a flowchart illustrating an exemplary search range determination processing flow of the information processing apparatus according to the first embodiment;

FIG. 9 is a diagram illustrating an exemplary structure of the functional blocks of an information processing apparatus according to a second embodiment;

FIG. 10 is a flowchart illustrating an exemplary onset time identification processing flow of the information processing apparatus in the second embodiment; and

FIG. 11 is a flowchart illustrating an exemplary onset search processing flow of the information processing apparatus according to a third embodiment.

The accompanying drawings are intended to depict exemplary embodiments of the present invention and should not be interpreted to limit the scope thereof. Identical or similar reference numerals designate identical or similar components throughout the various drawings.

### DESCRIPTION OF THE EMBODIMENTS

The terminology used herein is for the purpose of describing particular embodiments only and is not intended to be limiting of the present invention.

As used herein, the singular forms “a”, “an” and “the” are intended to include the plural forms as well, unless the context clearly indicates otherwise.

In describing preferred embodiments illustrated in the drawings, specific terminology may be employed for the sake of clarity. However, the disclosure of this patent specification is not intended to be limited to the specific terminology so selected, and it is to be understood that each specific element includes all technical equivalents that have the same function, operate in a similar manner, and achieve a similar result.

An embodiment of the present invention will be described in detail below with reference to the drawings.

An embodiment has an object to provide a biological signal analyzing apparatus, a waveform generation identification method, and a computer-readable medium that can automatically identify the time at which a signal is generated from an epileptic lesion, thereby making it possible to achieve efficient diagnosis for epilepsy and to increase the stability of diagnostic accuracy.

The following describes embodiments of a biological signal analyzing apparatus, a waveform generation identification method, and a computer program according to the present invention in detail with reference to the drawings. The invention is not limited by the following embodiments, and the components in the following embodiments include those readily conceivable by those who skilled in the art, those substantially identical, and those what are called equivalents. Furthermore, various omissions, substitutions, changes, and combinations of components may be made without departing from the gist of the following embodiments.

### First Embodiment

### Overall Structure of Biological Signal Measurement System

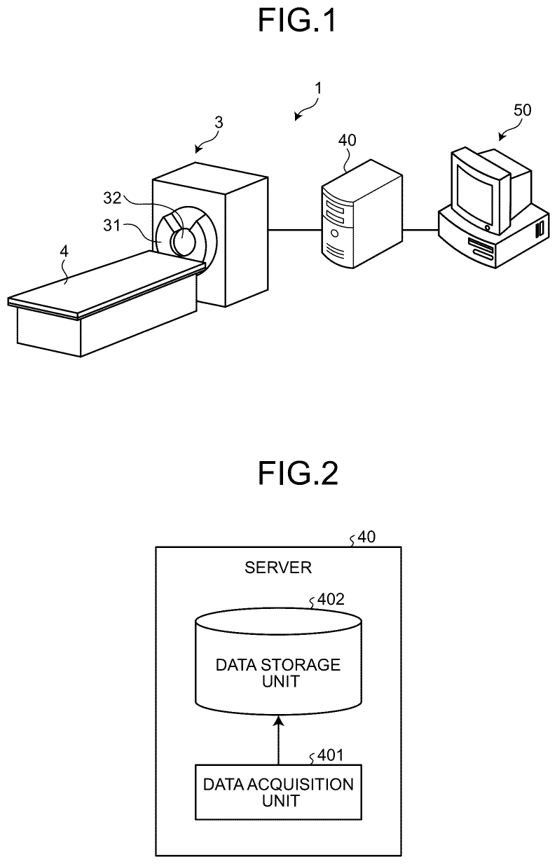

FIG. 1 is a diagram illustrating an exemplary overall structure of a biological signal measurement system in a first embodiment. FIG. 2 is a diagram illustrating an exemplary structure of functional blocks of a server in the first embodiment. The overall structure of this biological signal measurement system 1 according to the first embodiment is described with reference to FIGS. 1 and 2.

The biological signal measurement system 1 is a system that measures and displays a plurality of types of biological signals (e.g., magnetoencephalography (MEG) signals, electroencephalography (EEG) signals) coming from a specific signal source (body region) of a subject being tested. The biological signals to be measured are not limited to the MEG and EEG signals. The biological signals to be measured may be electrical signals generated in response to heart activity (electrical signals that can be expressed as electrocardiograms), for example.

As illustrated in FIG. 1, the biological signal measurement system 1 includes a measurement device 3 that measures one or more biological signals of a subject being tested, a server 40 that records one or more types of biological signals measured by the measurement device 3, and an information processing apparatus 50 that is a biological signal analyzing apparatus and analyzes one or more biological signals recorded in the server 40. The measurement device 3 is a magnetoencephalography that measures the MEG signals (an example of the biological signals) generated by cerebral magnetic fields or at the timing of applying stimulation, for example. FIG. 1 illustrates the server 40 and the information processing apparatus 50 separately. However, at least some of the functions of the server 40 may be incorporated into the information processing apparatus 50, for example.

In the example illustrated in FIG. 1, the subject being tested (the person being measured) lies down in a supine position on a measurement table 4 with electrodes (or sensors) for EEG measurement attached to his or her head and then puts his or her head in a recessed portion 32 of a dewar 31 of the measurement device 3. The dewar 31 is a holding vessel having a cryogenic environment with liquid helium. On the inside of the recessed portion 32 of the dewar 31, a number of magnetic sensors (e.g., superconducting quantum interference device (SQUID) sensors) for MEG measurement are arranged. The measurement device 3 collects the EEG signals from the electrodes and MEG signals from the magnetic sensors, and outputs time-series waveform data including the collected EEG signals and MEG signals (hereinafter the time-series waveform data is simply referred to as the waveform data) to the server 40. The waveform data output to the server 40 is read out to and displayed on the information processing apparatus 50 for analysis. Generally, the dewar 31, which has built-in magnetic sensors, and the measurement table 4 are installed in a magnetically shielded room. However, the magnetically shielded room is not illustrated in FIG. 1, as a matter of convenience.

The information processing apparatus 50 analyzes the waveform data of the MEG signals from the multiple magnetic sensors and the waveform data of the EEG signals from the multiple electrodes. For example, the information processing apparatus 50 displays the waveform data of the MEG signals and the EEG signals in a synchronized manner on the same time axis. The EEG signal expresses the electrical activity of neurons (the flow of ionic charge that occurs in the dendrites of the neurons during synaptic transmission) as a voltage value between the electrodes. The MEG signal expresses a minute fluctuation in the electric field caused by electrical activity in the brain.

The server 40 has a data acquisition unit 401 and a data storage unit 402, as illustrated in FIG. 2.

The data acquisition unit 401 is a functional unit that periodically acquires the waveform data such as the MEG signals and the EEG signals that are measured by the measurement device 3. The waveform data includes the time series data measured by the magnetic sensors arranged inside the dewar 31 of the measurement device 3 and the time series data of each of the EEG signals measured by the electrodes for EEG measurement attached to the head of the subject being tested (the person being measured).

The data storage unit 402 is a functional unit that stores therein the waveform data acquired from the measurement device 3.

FIG. 1 illustrates the structure in which the measurement device 3 is directly connected to the server 40 and the server 40 is directly connected to the information processing apparatus 50. The structure may be employed in which they can perform data communication among them via a network. The network connection can be achieved either in a wired manner or a wireless manner. The server 40 may also be a server on the network, for example, and may be connected by a cloud connection.

### Hardware Structure of Information Processing Apparatus

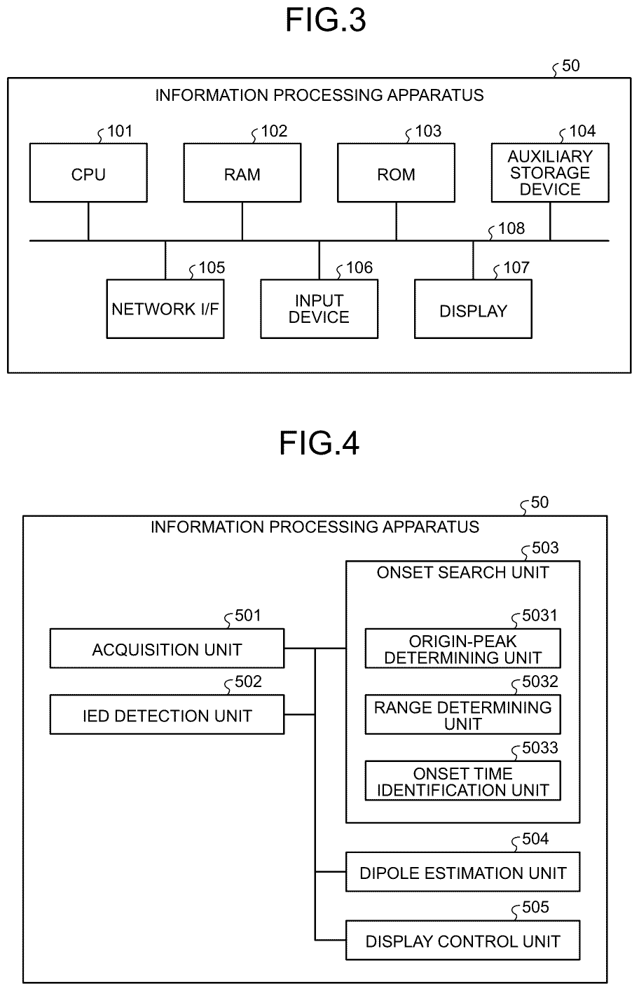

FIG. 3 is a diagram illustrating an exemplary hardware structure of the information processing apparatus according to the first embodiment. The hardware structure of the information processing apparatus 50 according to the first embodiment is described with reference to FIG. 3.

As illustrated in FIG. 3, the information processing apparatus 50 includes a central processing unit (CPU) 101, a random access memory (RAM) 102, a read only memory (ROM) 103, an auxiliary storage device 104, a network interface (I/F) 105, an input device 106, and a display 107.

The CPU 101 is an arithmetic unit that controls the operation of the entire information processing apparatus 50. The RAM 102 is a volatile storage device used as a work area for the CPU 101. The ROM 103 is a nonvolatile storage device that stores a program for the information processing apparatus 50.

The auxiliary storage device 104 is a storage device such as a hard disk drive (HDD) or a solid state drive (SSD) that stores various types of data and programs.

The network I/F 105 is an interface for data communication with external devices such as the server 40 via a network. The network I/F 105 is a network interface card (NIC) that is compatible with Ethernet (registered trademark) and capable of wired or wireless communication compliant with a transmission control protocol (TCP) or an Internet protocol (IP), for example.

The input device 106 includes an input function of a touch panel, a mouse, and a keyboard that are used for selecting letters, numbers, and various instructions, and for moving a cursor, for example.

The display 107 displays various types of information such as the cursor, menus, windows, letters, and images and is composed of liquid crystal, organic electro luminescence (EL), or the like.

The CPU 101, the RAM 102, the ROM 103, the auxiliary storage device 104, the network I/F 105, the input device 106, and the display 107 described above are coupled with a bus 108 such as an address bus and a data bus to communicate with one another.

The hardware structure of the information processing apparatus 50 illustrated in FIG. 3 is an example and need not include all of the components illustrated in FIG. 3, or may include other components.

### Structure and Operations of Functional Blocks of Information Processing Apparatus

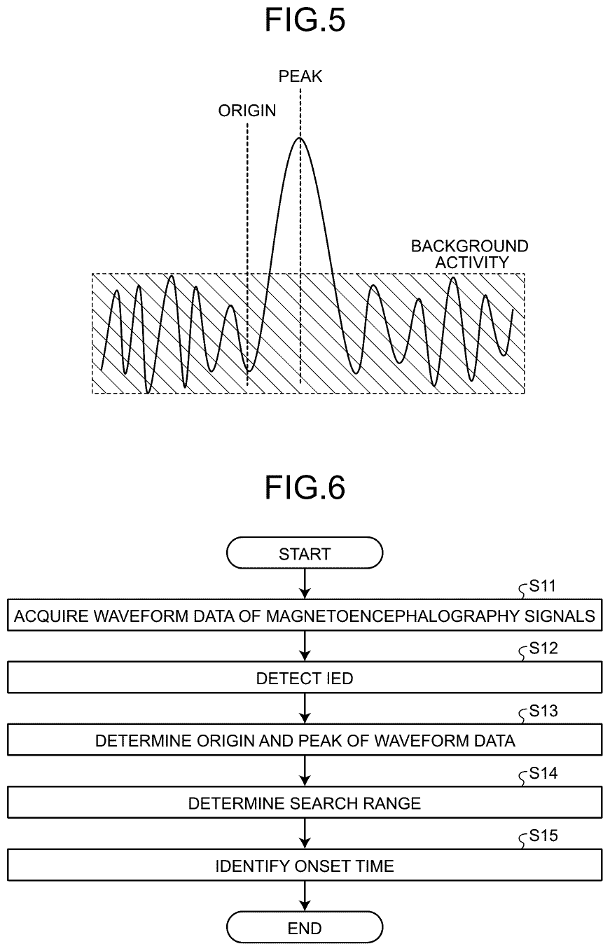

FIG. 4 is a diagram illustrating an exemplary structure of the functional blocks of the information processing apparatus according to the first embodiment. FIG. 5 is a diagram illustrating exemplary waveform data in which characteristic waveform information about the IED appears. The following describes the structure and operations of the functional blocks of the information processing apparatus 50 according to the first embodiment with reference to FIGS. 4 and 5.

As illustrated in FIG. 4, the information processing apparatus 50 has an acquisition unit 501, an IED detection unit 502 (detection unit), an onset search unit 503 (search unit), a dipole estimation unit 504, and a display control unit 505.

The acquisition unit 501 is a functional unit that acquires the waveform data of the MEG signals and the EEG signals from the server 40 via the network I/F 105. Among the waveform data, the waveform data of the MEG signals is focused to be described. The waveform data acquired by the acquisition unit 501 may be any of the waveform data measured by the measurement device 3, the waveform data stored in the data storage unit 402 of the server 40, or the waveform data stored in the auxiliary storage device 104 of the information processing apparatus 50.

The IED detection unit 502 is a functional unit that detects the time manually specified by, for example, an expert such as a doctor as the time at which the characteristic waveform information about the IED appears in the waveform data acquired by the acquisition unit 501. For example, an expert or the like checks the waveform data that corresponds to each sensor and is displayed on the display 107, determines the time at which the characteristic waveform information about the IED appears by visual observation, and performs the specification operation on the time via the input device 106. The IED detection unit 502 detects, as the time at which the characteristic waveform information about the IED appears, the time specified by the specification operation.

A concrete example of the waveform data in which the characteristic waveform information about the IED appears is illustrated in FIG. 5. The user, such as the expert, specifies the time at the center of the characteristic waveform information (a spike waveform) about the IED, i.e., the time considered to correspond to the peak of the spike waveform illustrated in FIG. 5. The peak refers to the apex of the characteristic waveform of the IED, and the origin illustrated in FIG. 5 refers to the part from which the mountain-like shape of the waveform begins. The background activity illustrated in FIG. 5 is defined as the waveform portion in normal brain activity other than characteristic waveforms of epileptic origin such as epileptic seizures or interictal epileptic discharges, or the waveform portion having a noise signal magnitude in the normal brain activity.

The onset search unit 503 is a functional unit that searches for an onset on the basis of the time (time at the peak) detected by the IED detection unit 502. The onset is defined as the portion in which the IED actually occurred (the signal is generated from the actual epileptic lesion) in the waveform data. Specifically, the onset search unit 503 identifies the time of onset (an onset time) on the basis of the time (time at the peak) detected by the IED detection unit 502. The onset search unit 503 has an origin-peak determining unit 5031 (first determining unit), a range determining unit 5032 (second determining unit), and an onset time identification unit 5033 (identification unit), as illustrated in FIG. 4.

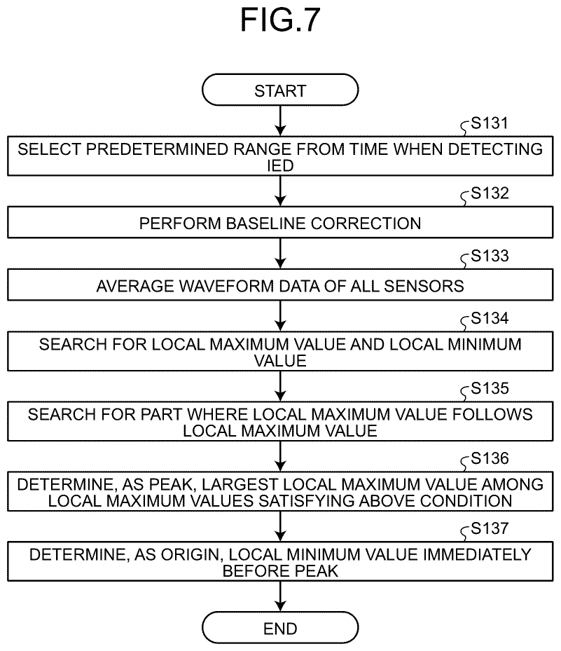

The origin-peak determining unit 5031 is a functional unit that determines the origin and the peak in the waveform data in which the characteristic waveform information about the IED appears on the basis of the time (the peak time) detected by the IED detection unit 502. The determination processing of the origin and the peak by the origin-peak determining unit 5031 is described later in detail in FIG. 7.

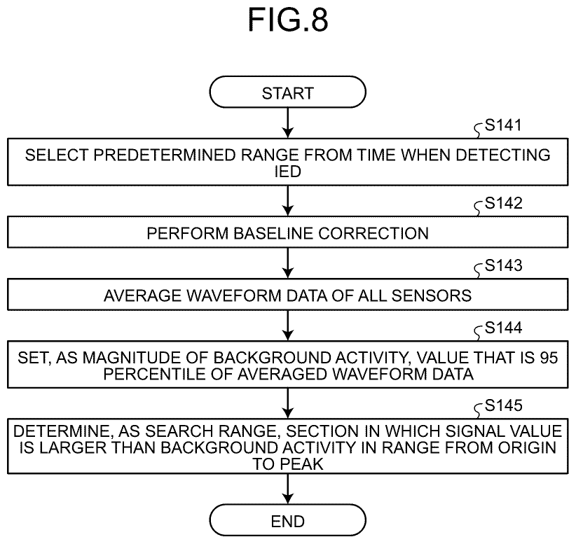

The range determining unit 5032 is a functional unit that determines, as a search range, a section in which a signal intensity is greater than the background activity in the range from the origin to the peak, the range being determined by the origin-peak determining unit 5031. The determination processing of the search range by the range determining unit 5032 is described later in detail in FIG. 8.

The onset time identification unit 5033 is a functional unit that identifies, as the onset time, the earliest time in the search range determined by the range determining unit 5032.

The dipole estimation unit 504 is a functional unit that performs dipole estimation using the waveform data at the onset time searched by the onset search unit 503.

The display control unit 505 is a functional unit that performs the display operation of the display 107. The display control unit 505 displays the waveform data acquired by the acquisition unit 501, in-process contents and the processing result by the onset search unit 503, and the estimation result by the dipole estimation unit 504.

The acquisition unit 501, the IED detection unit 502, the onset search unit 503, the dipole estimation unit 504, and the display control unit 505 are achieved by the CPU 101 illustrated in FIG. 3 executing the program. Some or all of the functions of the acquisition unit 501, the IED detection unit 502, the onset search unit 503, the dipole estimation unit 504, and the display control unit 505 may be achieved not by the program, which is software, but by a hardware circuit (integrated circuit) such as a field programmable gate array (FPGA) or an application specific integrated circuit (ASIC).

Each functional unit of the information processing apparatus 50 illustrated in FIG. 4 represents the function conceptually. The structure of the information processing apparatus 50 is not limited to that illustrated in FIG. 4. For example, the multiple functional units illustrated as independent functional units in the information processing apparatus 50 illustrated in FIG. 4 may be achieved as a single functional unit. The function of the functional unit in the information processing apparatus 50 illustrated in FIG. 4 may be divided into multiple functions and achieved by multiple functional units.

### Onset Search Processing Flow of Information Processing Apparatus

FIG. 6 is a flowchart illustrating an exemplary onset search processing flow of the information processing apparatus according to the first embodiment. FIG. 7 is a flowchart illustrating an exemplary origin-peak determination processing flow of the information processing apparatus according to the first embodiment. FIG. 8 is a flowchart illustrating an exemplary search range determination processing of the information processing apparatus according to the first embodiment. The following describes the flow of the onset search processing of the information processing apparatus 50 according to the first embodiment with reference to FIGS. 6 to 8.

### Step S11

The acquisition unit 501 acquires the waveform data of the MEG signals from the server 40 via the network I/F 105. Then, the processing proceeds to step S12.

### Step S12

The IED detection unit 502 detects the time manually specified by the expert such as a doctor, for example, as the time at which the characteristic waveform information of the IED appears in the waveform data acquired by the acquisition unit 501. For example, the expert or the like checks the waveform data that corresponds to each sensor and is displayed on the display 107, determines the time at which the characteristic waveform information about the IED appears by visual observation, and specifies the time by the specification operation via the input device 106. The IED detection unit 502 detects, as the time at which the characteristic waveform information about the IED appears, the time specified by the specification operation. Then, the processing proceeds to step S13.

### Step S13

The origin-peak determining unit 5031 of the onset search unit 503 determines the origin and the peak in the waveform data in which the characteristic waveform information about the IED appears on the basis of the time detected by the IED detection unit 502. Specifically, the origin-peak determining unit 5031 performs the processing illustrated at steps S131 to S137 illustrated in FIG. 7.

### Step S131

The origin-peak determining unit 5031 first selects a predetermined range (e.g., a range of ±40 (msec) centering on the time at the peak) on the basis of the time detected by the IED detection unit 502. Then, the processing proceeds to step S132.

### Step S132

The origin-peak determining unit 5031 performs baseline correction (or high-pass filtering or the like) on the waveform data with respect to all channels (i.e., all sensors) within the selected range. Then, the processing proceeds to step S133.

### Step S133

The origin-peak determining unit 5031 performs average processing on the waveform data of all channels (i.e., all sensors) after the baseline correction. The waveform data may be subjected to the average processing for all channels, or the absolute value of the waveform data may be subjected to the average processing for all channels. Then, the processing proceeds to step S134.

### Step S134

The origin-peak determining unit 5031 searches for a local maximum value and a local minimum value from the averaged waveform data for each sensor. Then, the processing proceeds to step S135.

### Step S135

The origin-peak determining unit 5031 searches for combinations of the maximum and minimum values, the combination satisfying that the local maximum value follows the local minimum value in the chronological order, among the searched local maximum and local minimum values. If the combination in which the local maximum value follows the local minimum value in the chronological order is not searched, processing may be made using, as the peak, a certain time after the time detected by the IED detection unit 502 (e.g., 40 (msec) after the time at the peak). Then, the processing proceeds to step S136.

### Step S136

The origin-peak determining unit 5031 determines, as the peak, the largest local maximum value in the local maximum values each included in the corresponding searched combinations. Then, the processing proceeds to step S137.

### Step S137

The origin-peak determining unit 5031 determines, as the origin, the local minimum value immediately before the determined peak, i.e., the local minimum value that is in combination with the local maximum value having the largest value. If the time detected by the IED detection unit 502 is determined as the time at the peak, the origin may be determined retroactively from the peak time. Then, step S13 (FIG. 6) ends and the processing proceeds to step S14.

### Step S14

The range determining unit 5032 of the onset search unit 503 determines the search range to search for the onset on the basis of the peak and the origin that are determined by the origin-peak determining unit 5031. Specifically, the range determining unit 5032 performs processing at steps S141 to S145 illustrated in FIG. 8.

### Step S141

The range determining unit 5032 first selects a predetermined range (e.g., −2040 to −40 (msec) from the time at the peak) that is a certain time retroactively from the time detected by the IED detection unit 502. The predetermined range is selected in a certain time retroactively from the time detected by the IED detection unit 502. However, the predetermined range is not limited to be selected in this manner. The range may be selected from a certain time advanced from the time detected by the IED detection unit 502. The waveform data in the entire time period may be used for the predetermined range. Then, the processing proceeds to step S142.

### Step S142

The range determining unit 5032 performs the baseline correction on the waveform data of all channels (i.e., all sensors) within the selected range. Then, the processing proceeds to step S143.

### Step S143

The range determining unit 5032 performs the average processing on the waveform data of all channels (i.e., all sensors) after the baseline correction. The waveform data may be subjected to the average processing for all channels, or the absolute value of the waveform data may be subjected to the average processing for all channels. The range determining unit 5032 may also use the averaged waveform data processed by the origin-peak determining unit 5031 at step S133 (FIG. 7). The raw waveform data may be used as it is instead of the averaged waveform data. Then, the processing proceeds to step S144.

### Step S144

The range determining unit 5032 determines, as the magnitude of the background activity, the signal value the ratio of which in the cumulative frequency distribution of signal values at each time in the averaged waveform data reaches a predetermined threshold (e.g., 95 percentile). The maximum, median, mean, or the mean+2 SD (SD is standard deviation) of the averaged waveform data within the predetermined range may be determined as the magnitude of the background activity. Then, the processing proceeds to step S145.

### Step S145

The range determining unit 5032 determines, as the search range, a section in which the signal value is greater than that of the background activity in the range from the origin to the peak that are determined by the origin-peak determining unit 5031. The background data to be compared to the background activity may be averaged waveform data or raw waveform data measured from sensors. Then, step S14 (FIG. 6) ends and the processing proceeds to step S15.

### Step S15

The onset time identification unit 5033 of the onset search unit 503 identifies, as the onset time, the earliest time in the search range determined by the range determining unit 5032. The onset search processing ends.

The dipole estimation unit 504 performs the dipole estimation on the basis of the onset time identified by the onset search processing and the waveform data used for identifying the onset time. The dipole estimation processing can be performed by employing conventional methods.

As described above, in the information processing apparatus 50 according to the first embodiment, the acquisition unit 501 acquires the waveform data of the biological signals measured by the multiple sensors, the IED detection unit 502 detects the time at which the characteristic waveform information about the IED appears in the waveform data acquired by the acquisition unit 501, and the onset search unit 503 searches for the onset time at which the signal is generated from the epileptic lesion within a predetermined search range based on the time detected by the IED detection unit 502 in the waveform. This enables automatic identification of the time at which the signal is generated from the epileptic lesion, thereby making it possible to achieve efficient diagnosis for epilepsy and to increase the stability of diagnostic accuracy.

The onset search unit 503 has the origin-peak determining unit 5031, the range determining unit 5032, and the onset time identification unit 5033. The origin-peak determining unit 5031 determines the origin and the peak of the waveform data in which the characteristic waveform information appears on the basis of the time detected by the IED detection unit 502. The range determining unit 5032 determines, as the search range, the section in which the signal intensity is greater than that of the background activity of the waveform data in the range from the origin to the peak that are determined by the origin-peak determining unit 5031. The onset time identification unit 5033 identifies the onset time from the search range determined by the range determining unit 5032. The search range is determined on the basis of the magnitude of the background activity, thereby making it possible to increase accuracy of identifying the onset time.

### Second Embodiment

The following describes the biological signal measurement system 1 according to a second embodiment, focusing on the differences from the biological signal measurement system 1 according to the first embodiment, the first embodiment, the operation is described in which the earliest time in the determined search range is identified as the onset time. In the second embodiment, operation is described in which the onset time is identified on the basis of various index values estimated by the dipole estimation. The overall structure of the biological signal measurement system 1 and the hardware structure of the information processing apparatus in the second embodiment are the same as those described in the first embodiment.

### Structure and Operations of Functional Blocks of Information Processing Apparatus

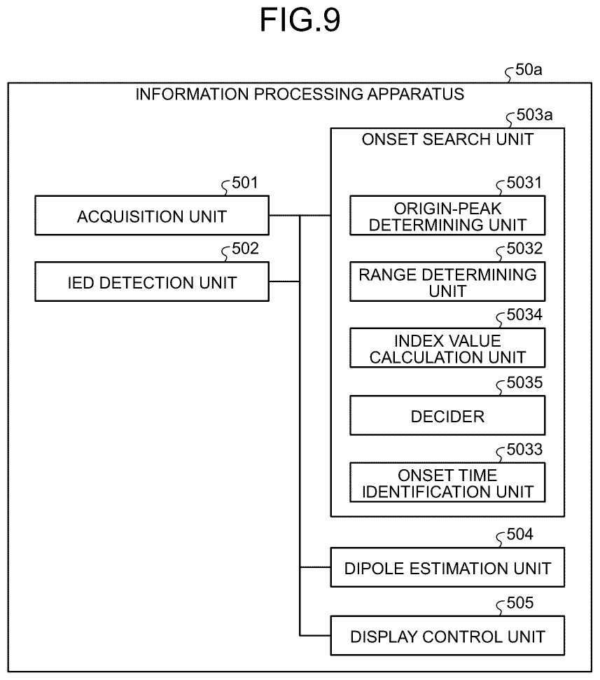

FIG. 9 is a diagram illustrating an exemplary structure of the functional blocks of the information processing apparatus according to the second embodiment. The following describes the structure and operations of the functional blocks of an information processing apparatus 50a according to the second embodiment with reference to FIG. 9.

As illustrated in FIG. 9, the information processing apparatus 50a has the acquisition unit 501, the IED detection unit 502 (detection unit), an onset search unit 503a (search unit), the dipole estimation unit 504, and the display control unit 505. The operations of the acquisition unit 501, the IED detection unit 502, the dipole estimation unit 504, and the display control unit 505 are the same as those of the information processing apparatus 50 according to the first embodiment.

The onset search unit 503a is a functional unit that searches for the onset on the basis of the time (time at the peak) detected by the IED detection unit 502. As illustrated in FIG. 9, the onset search unit 503a has the origin-peak determining unit 5031 (first determining unit), the range determining unit 5032 (second determining unit), an index value calculation unit 5034 (calculation unit), a decider 5035, and the onset time identification unit 5033 (identification unit). The operations of the origin-peak determining unit 5031 and the range determining unit 5032 are the same as those of the information processing apparatus 50 according to the first embodiment.

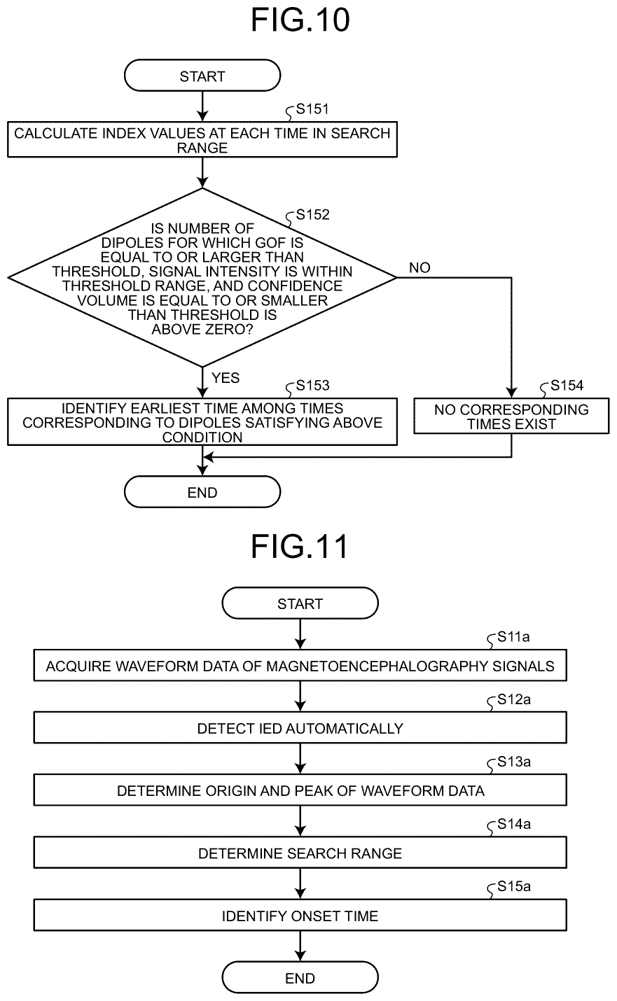

The index value calculation unit 5034 is a functional unit that calculates index values such as a goodness of fit (GOF), a signal strength, and a confidence volume, which are related to an estimation result, by the dipole estimation using waveform data at each time within the search range determined by the range determining unit 5032. The GOF is described in the article “Spatial resolution of neuromagnetic records: theoretical calculations in a spherical model (1988),” for example. The index value calculation processing by the index value calculation unit 5034 is described later in detail in FIG. 10.

The decider 5035 is a functional unit that decides whether the GOF is equal to or larger than a predetermined threshold, whether the signal intensity is within a predetermined threshold range, and the confidence volume is equal to or smaller than a predetermined threshold, within the search range determined by the range determining unit 5032. The GOF, the signal intensity, and the confidence volume are calculated by the index value calculation unit 5034 as the index values. The threshold for the GOF is 80%, the threshold range for the confidence intensity is from 0.5e⁻⁷ to 5e⁻⁷ (A), and the threshold value for the confidence volume is 2e⁻⁵ (m²), for example. The thresholds described above are examples. Other thresholds may be used.

The onset time identification unit 5033 identifies, as the onset time, the earliest time among the times that satisfy the conditions decided by the decider 5035 in the search range determined by the range determining unit 5032.

The acquisition unit 501, the IED detection unit 502, the onset search unit 503a, the dipole estimation unit 504, and the display control unit 505 are achieved by the CPU 101 illustrated in FIG. 3 executing a program. Some or all of the functions of the acquisition unit 501, the IED detection unit 502, the onset search unit 503a, the dipole estimation unit 504, and the display control unit 505 may be achieved not by the program, which is software, but by a hardware circuit (integrated circuit) such as the FPGA or the ASIC.

Each functional unit of the information processing apparatus 50a illustrated in FIG. 9 represents the function conceptually. The structure of the information processing apparatus 50a is not limited to that illustrated in FIG. 9. For example, the multiple functional units illustrated as independent functional units in the information processing apparatus 50a illustrated in FIG. 9 may be achieved as a single functional unit. The function of the functional unit in the information processing apparatus 50a illustrated in FIG. 9 may be divided into multiple functions and achieved by multiple functional units.

### Onset Search Processing Flow of Information Processing Apparatus

FIG. 10 is a flowchart illustrating an exemplary onset time identification processing flow of the information processing apparatus according to the second embodiment. The following describes the onset time identification processing in the onset search processing by the information processing apparatus 50a according to the second embodiment with reference to FIG. 10. In other words, the details of the processing at step S15 (FIG. 6) are described.

### Onset Time Identification Processing Flow

### Step S151

The index value calculation unit 5034 of the onset search unit 503a calculates index values related to the estimation result, such as the GOF, the signal strength, and the confidence volume by the dipole estimation using the waveform data at each time in the search range determined by the range determining unit 5032. The dipole estimation is not limited to being performed at all times within the search range. For example, the dipole estimation may be performed only in the first half of the search range, or at a time every other point in the search range. Then, the processing proceeds to step S152.

### Step S152

The decider 5035 of the onset search unit 503a decides whether the GOF is equal to or larger than a predetermined threshold, whether the signal intensity is within a predetermined threshold range, and the confidence volume is equal to or smaller than a predetermined threshold, within the search range determined by the range determining unit 5032. The GOF, the signal intensity, and the confidence volume are calculated by the index value calculation unit 5034 as the index values. If a dipole exists for which the GOF is equal to or larger than the predetermined threshold, the signal strength is within the predetermined threshold range, and the confidence volume is equal to or smaller than the predetermined threshold (Yes at step S152), the processing proceeds to step S153. If such dipole does not exist (No at step S152), the processing proceeds to step S154.

It is not necessary to satisfy all three conditions described above. For example, if two of the three conditions are satisfied, or if at least one of the three conditions is satisfied, the processing may proceed to step S153. The index values calculated by the index value calculation unit 5034 are not limited to the three index values described above, and other index values may be calculated.

### Step S153

The onset time identification unit 5033 of the onset search unit 503a identifies, as the onset time, the earliest time among the times that satisfy the conditions decided by the decider 5035 in the search range determined by the range determining unit 5032. The onset time identification processing ends.

### Step S154

If there is no time that satisfies the conditions decided by the decider 5035, the onset time identification unit 5033 ends the onset time identification processing because the onset time satisfying the conditions cannot be identified. If there is no time that satisfies the conditions decided by the decider 5035, a method that uses the time detected by the IED detection unit 502 as the onset time, a method that adopts the time halfway between the origin and the peak as the onset time, or the like can also be employed.

As described above, in the onset search unit 503a of the information processing apparatus 50a according to the second embodiment, the index value calculation unit 5034 calculates one or more index values related to the dipole estimation using the waveform data at each time within the search range determined by the range determining unit 5032, the decider 5035 decides whether the index values calculated by the index value calculation unit 5034 satisfy the predetermined conditions, and the onset time identification unit 5033 identifies, as the onset time, the earliest time among the times that satisfy the conditions decided by the decider 5035 in the search range. The decision is made whether the index values related to the dipole estimation for waveform data in the search range satisfy the predetermined conditions, and the earliest time among the times that satisfy the conditions is identified as the onset time, thereby making it possible to further increase the accuracy of identifying the onset time.

### Third Embodiment

The following describes the biological signal measurement system 1 according to a third embodiment, focusing on the differences from the biological signal measurement system 1 according to the first embodiment. In the first embodiment, the operation is described in which the time at which the characteristic waveform information about the IED appears is identified manually. In the third embodiment, the operation is described in which the time at which the characteristic waveform information about the IED appears is automatically identified using a machine learned model. The overall structure of the biological signal measurement system 1, the hardware structure of the information processing apparatus, and the structure of functional blocks according to the third embodiment are the same as those described in the first embodiment.

### Onset Search Processing Flow of Information Processing Apparatus

FIG. 11 is a flowchart illustrating an exemplary onset search processing flow of the information processing apparatus according to the third embodiment. The following describes the onset search processing flow of the information processing apparatus 50 according to the third embodiment with reference to FIG. 11.

### Step S11a

The acquisition unit 501 acquires the waveform data of the MEG signals from the server 40 via the network I/F 105. Then, the processing proceeds to step S12a.

### Step S12a

The IED detection unit 502 detects the time at which the characteristic waveform information about the IED appears in the waveform data acquired by the acquisition unit 501 using a machine learned model. For the learned model, a machine learned model described in Japanese Unexamined Patent Application Publication No. 2021-069929 can be employed, for example. Then, the processing proceeds to step S13a.

### Steps S13a to S15a

The processes at steps S13a to S15a are the same as those at steps S13 to S15 (FIG. 6), respectively. The onset search processing ends.

As described above, the IED detection unit 502 can detect the time automatically, thereby making it possible to automatically detect the time at which the characteristic waveform information about the IED appears in the waveform data, and identify the onset time.

In each of the embodiments, when at least one of the functional units of the information processing apparatuses 50 and 50a are achieved by execution of the program, the program is embedded and provided in a ROM, for example. In each of the embodiments, the program executed by the information apparatuses 50 and 50a may be recorded in a computer-readable recording medium such as a compact disc read only memory (CD-ROM), a flexible disk (FD), a compact disc recordable (CD-R), and a digital versatile disc (DVD), as an installable or executable file, and provided. In each of the embodiments, the program executed by the information apparatuses 50 and 50a may be stored in a computer connected to a network such as the Internet, and provided by being downloaded via the network. In each of the embodiments, the program executed by the information apparatuses 50 and 50a may be provided or distributed via a network such as the Internet. In each of the embodiments, the program executed by the information apparatuses 50 and 50a has a modular configuration including at least one of the functional units. As the actual hardware, the CPU 101 reads and executes the program from the storage device (e.g., the ROM 103, the auxiliary storage device 104, or the like). As a result, each of the functional units is loaded onto the main storage device (the RAM 102).

The aspects of the present invention are as follows:

<1> A biological signal analyzing apparatus including:
an acquisition unit configured to acquire waveform data of biological signals measured by a plurality of sensors;
a detection unit configured to detect a time at which characteristic waveform information about an interictal epileptiform discharge (IED) appears in the waveform data acquired by the acquisition unit; and
a search unit configured to search for an onset time at which a signal is generated from a lesion in a brain within a predetermined search range based on the time detected by the detection unit in the waveform data.
   <2> The biological signal analyzing apparatus according to <1>, wherein the search unit includes:
a first determining unit configured to determine an origin and a peak of the waveform data in which the characteristic waveform information appears, based on the time detected by the detection unit;
a second determining unit configured to determine, as the search range, a section in which a signal intensity is greater than a signal intensity of a background activity of the waveform data in a range from the origin to the peak determined by the first determining unit; and
an identification unit configured to identify the onset time from the search range determined by the second determining unit.
   <3> The biological signal analyzing apparatus according to <2>, wherein the identification unit is configured to identify an earliest time in the search range as the onset time.
   <4> The biological signal analyzing apparatus according to <2>, wherein the search unit further includes:
a calculation unit configured to calculate one or more index values related to dipole estimation using the waveform data at each time within the search range determined by the second determining unit; and
a decider configured to decide whether the one or more index values calculated by the calculation unit satisfy a predetermined condition, and
the identification unit is configured to identify, as the onset time, an earliest time among times at which the decider decides that the condition is satisfied.
   <5> The biological signal analyzing apparatus according to <4>, wherein the calculation unit is configured to calculate at least any one of a good of fitness (GOF), a confidence intensity, and a confidence volume as each of the one or more index values by the dipole estimation using the waveform data at each time within the search range determined by the second determining unit.
   <6> The biological signal analyzing apparatus according to any one of <2> to <5>, wherein the first determining unit is configured to determine the origin and the peak in data obtained by averaging the waveform data acquired by the acquisition unit.
   <7> The biological signal analyzing apparatus according to any one of <2> to <5>, wherein the second determining unit is configured to calculate the background activity from data obtained by averaging the waveform data acquired by the acquisition unit, and determine the search range using the background activity.
   <8> The biological signal analyzing apparatus according to any one of <1> to <5>, wherein the detection unit is configured to detect a time specified via an input device as the time at which the characteristic waveform information appears in the waveform data acquired by the acquisition unit.
   <9> The biological signal analyzing apparatus according to any one of <1> to <5>, wherein the detection unit is configured to detect the time at which the characteristic waveform information appears from the waveform data acquired by the acquisition unit, using a machine learned model.
   <10> A waveform generation identification method including:
acquiring waveform data of biological signals measured by a plurality of sensors;
detecting a time at which characteristic waveform information about an interictal epileptiform discharge (IED) appears in the acquired waveform data; and
searching for an onset time at which a signal is generated from a lesion in a brain in a predetermined search range based on the detected time in the waveform data.
   <11> A computer program for causing a computer to perform:
acquiring waveform data of biological signals measured by a plurality of sensors;
detecting a time at which characteristic waveform information about an interictal epileptiform discharge (IED) appears in the acquired waveform data; and searching for an onset time at which a signal is
generated from a lesion in a brain in a predetermined search range based on the detected time in the waveform data.

According to the present invention, the time at which the signal is generated from the lesion in the brain can be automatically identified, thereby making it possible to efficient diagnosis for epilepsy and to increase the stability of diagnostic accuracy.

The above-described embodiments are illustrative and do not limit the present invention. Thus, numerous additional modifications and variations are possible in light of the above teachings. For example, at least one element of different illustrative and exemplary embodiments herein may be combined with each other or substituted for each other within the scope of this disclosure and appended claims. Further, features of components of the embodiments, such as the number, the position, and the shape are not limited the embodiments and thus may be preferably set. It is therefore to be understood that within the scope of the appended claims, the disclosure of the present invention may be practiced otherwise than as specifically described herein.

The method steps, processes, or operations described herein are not to be construed as necessarily requiring their performance in the particular order discussed or illustrated, unless specifically identified as an order of performance or clearly identified through the context. It is also to be understood that additional or alternative steps may be employed.

Further, any of the above-described apparatus, devices or units can be implemented as a hardware apparatus, such as a special-purpose circuit or device, or as a hardware/software combination, such as a processor executing a software program.

Further, as described above, any one of the above-described and other methods of the present invention may be embodied in the form of a computer program stored in any kind of storage medium. Examples of storage mediums include, but are not limited to, flexible disk, hard disk, optical discs, magneto-optical discs, magnetic tapes, nonvolatile memory, semiconductor memory, read-only-memory (ROM), etc.

Alternatively, any one of the above-described and other methods of the present invention may be implemented by an application specific integrated circuit (ASIC), a digital signal processor (DSP) or a field programmable gate array (FPGA), prepared by interconnecting an appropriate network of conventional component circuits or by a combination thereof with one or more conventional general purpose microprocessors or signal processors programmed accordingly.

Each of the functions of the described embodiments may be implemented by one or more processing circuits or circuitry. Processing circuitry includes a programmed processor, as a processor includes circuitry. A processing circuit also includes devices such as an application specific integrated circuit (ASIC), digital signal processor (DSP), field programmable gate array (FPGA) and conventional circuit components arranged to perform the recited functions.

What is claimed is:

1. A biological signal analyzing apparatus comprising:
an acquisition unit configured to acquire waveform data of biological signals measured by a plurality of sensors;
a detection unit configured to detect a time at which characteristic waveform information about an interictal epileptiform discharge (IED) appears in the waveform data acquired by the acquisition unit; and
a search unit configured to search for an onset time at which a signal is generated from a lesion in a brain within a predetermined search range based on the time detected by the detection unit in the waveform data.

2. The biological signal analyzing apparatus according to claim 1, wherein the search unit includes:
a first determining unit configured to determine an origin and a peak of the waveform data in which the characteristic waveform information appears, based on the time detected by the detection unit;
a second determining unit configured to determine, as the search range, a section in which a signal intensity is greater than a signal intensity of a background activity of the waveform data in a range from the origin to the peak determined by the first determining unit; and
an identification unit configured to identify the onset time from the search range determined by the second determining unit.

3. The biological signal analyzing apparatus according to claim 2, wherein the identification unit is configured to identify an earliest time in the search range as the onset time.

4. The biological signal analyzing apparatus according to claim 2, wherein the search unit further includes:
a calculation unit configured to calculate one or more index values related to dipole estimation using the waveform data at each time within the search range determined by the second determining unit; and
a decider configured to decide whether the one or more index values calculated by the calculation unit satisfy a predetermined condition, and
the identification unit is configured to identify, as the onset time, an earliest time among times at which the decider decides that the condition is satisfied.

5. The biological signal analyzing apparatus according to claim 4, wherein the calculation unit is configured to calculate at least any one of a good of fitness (GOF), a confidence intensity, and a confidence volume as each of the one or more index values by the dipole estimation using the waveform data at each time within the search range determined by the second determining unit.

6. The biological signal analyzing apparatus according to claim 2, wherein the first determining unit is configured to determine the origin and the peak in data obtained by averaging the waveform data acquired by the acquisition unit.

7. The biological signal analyzing apparatus according to claim 2, wherein the second determining unit is configured to calculate the background activity from data obtained by averaging the waveform data acquired by the acquisition unit, and determine the search range using the background activity.

8. The biological signal analyzing apparatus according to claim 1, wherein the detection unit is configured to detect a time specified via an input device as the time at which the characteristic waveform information appears in the waveform data acquired by the acquisition unit.

9. The biological signal analyzing apparatus according to claim 1, wherein the detection unit is configured to detect the time at which the characteristic waveform information appears from the waveform data acquired by the acquisition unit, using a machine learned model.

10. A waveform generation identification method comprising:
acquiring waveform data of biological signals measured by a plurality of sensors;
detecting a time at which characteristic waveform information about an interictal epileptiform discharge (IED) appears in the acquired waveform data; and
searching for an onset time at which a signal is generated from a lesion in a brain in a predetermined search range based on the detected time in the waveform data.

11. A computer program for causing a computer to perform:
acquiring waveform data of biological signals measured by a plurality of sensors;
detecting a time at which characteristic waveform information about an interictal epileptiform discharge (IED) appears in the acquired waveform data; and
searching for an onset time at which a signal is generated from a lesion in a brain in a predetermined search range based on the detected time in the waveform data.
